(19) United States
(12) Patent Application Publication
(10) Pub. No.: US 2004/0053387 A1
(43) Pub. Date: Mar. 18, 2004

(54) Sieve tube-localized glutathione S-transferase

(75) Inventors: Hiroaki Hayashi, Kanagawa (JP); Akari Fukuda, Akita (JP); Tsutomu Shimuzu, Shizuoka (JP)

Correspondence Address: FOLEY AND LARDNER, SUITE 500, 3000 K STREET NW, WASHINGTON, DC, 20007, US

(73) Assignee: KUMIAI CHEMICAL INDUSTRY CO., LTD.

(21) Appl. No.: 10/390,184

(22) Filed: Mar. 18, 2003

(30) Foreign Application Priority Data
Sep. 13, 2002 (JP) 268680/2002

(51) Int. Cl. C12N 9/10; A01H 1/00; C12N 15/82; C12N 5/04

(52) U.S. Cl. 435/193; 435/419; 435/320.1; 800/290

(57) ABSTRACT

This invention provides a sieve tube-localized glutathione S-transferase by analyzing proteins existing in the phloem sap of rice plants and identifying proteins highly homologous to glutathione S-transferases of plants.

### FIELD OF THE INVENTION

The present invention relates to a sieve tube-localized glutathione S-transferase.

### BACKGROUND OF THE INVENTION

In higher plants, the phloem is a long-distance transport pathway for metabolites and signals. A main component of the phloem is a sieve tube, which comprises a series of cells referred to as sieve elements. The sieve element has lost most of its organelles (e.g., nucleus, vacuole, and Golgi apparatus) and ribosomes at the time of differentiation (Cronshaw, Ann. Rev. Plant Physiol. 32: 465484, 1981). Sieve elements, which lack nuclei and others, are considered to be incapable of synthesizing proteins (Oparka et al., Annu. Rev. Plant Physiol. Plant Mol. Biol. 51: 323-47, 2000). However, proteins are necessary in order to maintain physiological functions of the sieve tube. Thus, it is considered that proteins are supplied via the protoplasmic pathway to the sieve element through a companion cell adjacent thereto. In fact, 100 or more types of proteins have been detected in phloem sap, which is a sieve tube content of Cucurbit, wheat, Ricinus, and rice plants (Eschrich and Heyser, Encyclopedia of Plant Physiology, N. S. vol. 1: Transport in Plants, 1. Phloem transport, eds. M. H. Zimmermarn and J. A. Milburn, pp. 101-136, Springer, Berlin, 1975; Fisher et al., Plant Physiol. 100: 1433-1441, 1992; Sakuth et al., Planta 191: 207-213, 1993; Nakamura et al., Plant Cell Physiol. 34, 927-933, 1993).

Pumpkins contain highly concentrated P-proteins in their phloem sap, and among P-proteins, PP1 and PP2 have been analyzed (Clark et al., Plant J. 12, 49-61, 1997; Bostwick et al., Plant Mol. Biol. 26, 887-897, 1994). It has been demonstrated that in pumpkins, the phloem sap protein CmPP16 which binds to an antibody against a virus movement protein, is capable of transporting MRNA among cells (Xoconostle-Cazares et al., Science 283: 94-98, 1999). Further, housekeeping proteins, i.e., ubiquitins and molecular chaperones, have been detected in the phloem sap of Ricinus (Schobert et al., Planta 196, 205-210, 1995). This suggests the occurrence of turnover and folding of proteins in the sieve element. Thioredoxin h and glutaredoxin have been detected in phloem sap of rice plants and Ricinus, respectively (Ishiwatari et al., Planta 195, 456-463, 1995; Szederkenyi et al., Planta 202, 349-356, 1997). These two types of phloem sap proteins are thiol-redox proteins, and the reduction of the S—S bond is considered to repair proteins that were damaged by oxidation (Ishiwatari et al., Planta 195, 456-463, 1995; Szederkenyi et al., Planta 202, 349-356, 1997). Also, the enzyme activity of protein phosphorylation has been detected in the phloem sap of rice plants. This suggests the existence of a calcium signal transduction cascade in the sieve tube (Nakamuraet al., Plant Cell Physiol.34, 927-933,1993). However, only a few of the phloem proteins have been identified, and most of the soluble proteins have not yet been identified or analyzed.

On the other hand, glutathione S-transferase (hereinafter it is referred to as “GST”) catalyzes the conjugation of tripeptide glutathione (γ-Glu-Cys-Gly; GSH) with various hydrophobic, electrophilic compounds. Plant GSTs are classified into three groups (type I, type II, and type III) based on their amino acid sequences and intron/exon structures (Droog, J. Plant Growth Regul. 16: 95-107,1997). Regarding the functions of these plant GSTs, their ability to detoxify herbicides has drawn the most attentions (Marrs, Annu. Rev. Plant Physiol. Plant Mol. Biol. 47, 127-158, 1996). Specifically, plant GSTs allow GSH to conjugate with herbicides, thereby changing them into more hydrophilic, less toxic forms. This form of herbicide is sequestered in vacuoles. Further, it is reported that plant GSTs are induced by stresses (e.g., application of dehydration, damage, active oxygen, pathogen challenge, and xenobiotics) (Kiyosue et al., FEBS Lett. 335: 189-192, 1993; Kim et al., Plant Cell Rep. 13:341-343,1994; Bartling et al., Eur. J. Biochem. 216, 579-586,1993; Dudler et al., Mol. Plant-Microbe Interact. 4, 14-18, 1991; Wiegand et al., Plant Mol. Biol. 7,235-243,1986). These stress-inducible GSTs allow GSH to conjugate with reactive molecules, thereby detoxifying them. Accordingly, the stress-inducible GSTs, together with the aforementioned thioredoxin h and glutaredoxin, are considered to protect cells from oxidative damage (Marrs, Annu. Rev. Plant Physiol. Plant Mol. Biol. 47, 127-158, 1996). However, research on GSTs in normal plants is limited to the aforementioned ability to detoxify herbicides, sequestration of anthocyanin in vacuoles (Marrs et al., Nature 375, 397400, 1995; Alfenito et al., Plant Cell 10, 1135-1149, 1998), and the auxin-binding activity (Bilang and Sturm, Plant Physiol. 109, 253-260, 1995). Moreover, distribution of GSTs in normal plant tissues has not been sufficiently analyzed.

Highly concentrated (about 4 mM) reduced GSH has been detected in phloem sap of rice (Kuzuhara et al., Soil Sci. Plant Nutr. 46, 265-270, 2000). However, GST was not identified in the phloem sap of rice.

### SUMMARY OF THE INVENTION

An object of the present invention is to provide, for example, a sieve tube-localized glutathione S-transferase by identifying and analyzing proteins existing in the sieve tube of rice.

As a result of intensive and extensive studies aimed at the solution of the aforementioned objective, the present invention was completed through the analysis of proteins that exist in phloem sap and through the identification of a protein that is highly homologous to plant glutathione S-transferases.

More specifically, the present invention relates to a gene encoding a protein described in the following (a) or (b):

(a) a protein consisting of the amino acid sequence as shown in SEQ ID NO: 1; or

(b) a protein consisting of the amino acid sequence having the substitution, deletion, or addition of at least one amino acid in the amino acid sequence as shown in SEQ ID NO: 1 and having glutathione S-transferase activity.

The present invention relates to a sieve tube-localized glutathione S-transferase protein that is encoded by the gene.

The present invention also relates to a recombinant vector having the gene or the recombinant vector additionally having a companion cell-specific promoter located upstream of the gene.

The present invention further relates to a transformant having the recombinant vector having the gene.

The present invention relates to a plant body that has the recombinant vector additionally having a companion cell-specific promoter located upstream of the gene and that has resistance against agents.

The present invention is hereafter described in detail.

The gene of the present invention encodes a protein described in the following (a) or (b):

(a) a protein consisting of the amino acid sequence as shown in SEQ ID NO: 1; or

(b) a protein consisting of the amino acid sequence having the substitution, deletion, or addition of at least one amino acid in the amino acid sequence as shown in SEQ ID NO: 1 and having glutathione S-transferase activity.

A gene encoding the protein according to (a) encodes a sieve tube-localized glutathione S-transferase protein consisting of the amino acid sequence as shown in SEQ ID NO: 1 (hereinafter referred to as “sieve tube-localized GST protein”). The nucleotide sequences of the gene encoding the amino acid sequence as shown in SEQ ID NO: 1 are registered as two types of rice (O. sativa, cv. Nipponbare) EST clones, i.e., S13415 (Accession No. D 47745, SEQ ID NO: 2) and R2087 (Accession No. D24517, SEQ ID NO: 3), and the rice (O. sativa, cv. Nipponbare) BAC clone, i.e., OSJNBb0015I11 (Accession No. AC051633-22). The protein encoded by the gene (hereinafter referred to as “RPP31”) is highly analogous to type I plant GST. RPP31 has a Glu-Ser-Arg triplet. This triplet is highly conserved in the GSH binding site (G-site) of type I plant GST (Neuefeind et al., Biol. Chem. 378, 199-205, 1997; McGonigle et al., Plant Physiol. 124, 1105-1120, 2000). According to the result of DNA sequencing of the aforementioned BAC clone (OSJNBb0015I11 (Accession No. AC051633-22)), the gene encoding RPP31 contains 3 exons and 2 introns. This genetic structure is consistent with type I plant GST. Accordingly, RPP31 can be identified as the type I plant GST.

The term “sieve tube-localized” used herein refers to the phenomenon wherein a protein, which was expressed in the plant's companion cell, is transported to the adjacent sieve element and exists at least in the sieve tube (sieve element). Thus, RPP31 is localized not only in the companion cell but also localized in the sieve element. This can be confirmed by the immunohistological staining experiment on the cross-sectioned slice of the base of the leaf sheath of rice. The localization of RPP31 in the sieve tube can be confirmed regardless of the growth stage of the leaves.

The sieve element is a cell that constitutes a sieve tube, and it has lost most of its organelles (e.g., nucleus, vacuole, and Golgi apparatus) and ribosomes at the time of differentiation (Cronshaw, Ann. Rev. Plant Physiol. 32: 465484, 1981). Sieve elements, which lack nuclei and others, are considered to be incapable of synthesizing proteins (Oparka et al., Annu. Rev. Plant Physiol. Plant Mol. Biol. 51: 323-47, 2000). Thus, it is considered that proteins are supplied via the protoplasmic pathway to the sieve element through a companion cell adjacent thereto. Accordingly, RPP31 is considered to be effectively transported into the sieve element, which is incapable of synthesizing protein, through the companion cell. RPP31 exists in the organ containing the sieve tube. Examples of organs that contain the sieve tube involve leaves and stems of plants, and specifically leaves. Further, the presence of RPP31 in plant organs can be specified by subjecting each organ to Western blot analysis. For example, as a result of Western blot analysis, RPP31 is not detected in the root extract. This suggests that the sieve tube of the root contains no or amounts of RPP31 so small that the amount thereof is lower than the detection limit.

A gene encoding the protein according to (b) encodes a protein consisting of the amino acid sequence having the substitution, deletion, or addition of one or several (e.g., 1 to 10 or 1 to 5) amino acids in the amino acid sequence as shown in SEQ ID NO: 1 and having GST activity. The term “GST activity” refers to any activity that involves the conjugation of GSH with various hydrophobic, electrophilic compounds. An example of methods for assaying GST activity is the method in which a general GST substrate, 1-chloro-2,4-dinitrobenzene (CDNB), is used. With this method, a CDNB-GSH conjugate results from GST activity. The CDNB-GSH conjugate has an absorbance peak at 340 nm. Accordingly, an increase in the absorbance at 340 nm is measured as GST activity.

Once the nucleotide sequence of the gene of the present invention is established, the gene of the present invention can be then obtained by chemical synthesis, PCR using a cloned clone as a template, or hybridization using a DNA fragment having the nucleotide sequence as a probe. Further, a variant of the gene of the present invention having functions equivalent to those before the variation can be synthesized by site-directed mutagenesis, etc.

Examples of methods for introducing variation into the gene of the present invention include known methods such as the Kunkel method and the Gapped duplex method, or methods in accordance therewith. For example, variation is introduced using a kit for introducing variation which utilizes site-directed mutagenesis (e.g., Mutant-K (Takara) or Mutant-G (Takara)) or the LA PCR in vitro Mutagenesis Series Kit (Takara).

The sieve tube-localized GST protein of the present invention is encoded by the gene of the present invention. For example, the gene of the present invention is incorporated into a vector derived from Escherichia coli, etc., and Escherichia coli is then transformed using the resulting recombinant vector. Thereafter, the protein synthesized in Escherichia coli is extracted. Thus, the protein of the present invention can be obtained.

Further, the recombinant vector of the present invention has the gene of the present invention. Insertion of the gene of the present invention into a suitable vector can yield the recombinant vector of the present invention. Vectors for inserting the gene of the present invention are not particularly limited as long as they are replicable in the host. Examples thereof include a plasmid, a shuttle vector, and a helper plasmid, etc. When the vector is incapable of self-replication, a DNA fragment may become replicable upon, for example, insertion into the host chromosome.

Examples of plasmid DNA include: Escherichia coli-derived plasmids, for example, pET system such as pET30b, pBR system such as pBR322 and pBR325, pUC system such as pUC118, pUC119, pUC18 and pUC19, and pBluescript; Bacillus subtilis-derived plasmids such as pUB110 and pTP5; and yeast-derived plasmids, for example, YEp system such as YEpl3, and YCp system such as YCp50. Examples of phage DNA include λ phages such as Charon 4A, Charon 21A, EMBL3, EMBL4, λgt10, λgt11, and λZAP. Further, vectors of animal viruses such as retrovirus or vaccinia virus, vectors of plant viruses such as cauliflower mosaic virus, or vectors of insect viruses such as baculovirus can be used.

In order to insert the gene of the present invention into a vector, a method is employed in which cDNA of the gene of the present invention is first cleaved with a suitable restriction enzyme, and then inserted into the restriction enzyme site or multicloning site of a suitable vector DNA, thereby ligating to a vector. Alternatively, a method may be employed in which homologous regions are provided in a part of the vector and in a part of cDNA of the gene of the present invention, respectively, and they are ligated to each other by the in vitro method using PCR and the like or the in vivo method using yeast and the like.

The recombinant vector of the present invention further has a companion cell-specific promoter located upstream of the gene of the present invention. The term “companion cell-specific promoter” used herein refers to a promoter that specifically exhibits promoter activity in the plant's companion cell. An example of companion cell-specific promoters is, but is not limited to, thioredoxin h promoter of rice (Ishiwatari et al, Plant nutrition—for sustainable food production and environment. 809-810, 1997). A companion cell-specific promoter is inserted upstream of the gene of the present invention as follows. The promoter is inserted into a site where the promoter exhibits its activity and the gene of the present invention is expressed by the activity of the promoter. A companion cell-specific promoter may be inserted into a vector in the same manner as the method for introducing the gene of the present invention into the vector.

The transformant of the present invention has the recombinant vector of the present invention. The transformant of the present invention can be obtained by introducing the recombinant vector of the present invention into a host. Hosts are not particularly limited as long as the gene of the present invention can be expressed therein, and plants are preferred. When a host is a plant, transformed plants (transgenic plants) can be obtained in the following manner.

The “plants” which are to be transformed in the present invention refer to any of a whole plant, plant organs (e.g., leaves, flower petals, stems, roots, or seeds, etc.), plant tissues (e.g., epidermis, phloem, parenchyma, or xylem, etc.), or cultured cells of plants. Plants used in the transformation include, but are not limited to, those belonging to Gramineae, Brassicaceae, Solanaceae, or Leguminosae, etc (see below).

Gramineae: rice (Oryza sativa) and maize (Zea mays)

Brassicaceae: Arabidopsis thaliana

Solanaceae: tobacco plant (Nicotiana tabacum)

Leguminosae: soybean (Glycine max) The recombinant vector of the present invention can be introduced into plants by conventional transformation methods such as the electroporation method, the Agrobacterium method, the particle gun method, or the PEG method.

For example, when the electroporation method is employed, the recombinant vector of the present invention is introduced into the host using an electroporation apparatus equipped with a pulse controller under conditions of voltage of 500 to 1,600 V, 25 to 1,000 μF, and 20 to 30 msec.

When the particle gun method is employed, unchanged, after the preparation of slices or after the preparation of protoplast, the whole plant, plant organs, or plant tissues may be used. Subsequently, a gene introducing device such as PDS-1000/He of Bio-Rad can be used to treat the prepared samples. Although treatment conditions vary depending on types of plants or samples, it is generally carried out at the pressure of approximately 1,000 to 1,800 psi at a distance of approximately 5 to 6 cm.

In the method utilizing Ti plasmid or Ri plasmid of Agrobacterium, the gene of the present invention is introduced into a plant host by utilizing the characteristic that, upon infection of plants by bacteria belonging to the genus Agrobacterium, a part of the plasmid DNA of the bacteria is transferred into the plant genome. Among the bacteria belonging to the genus Agrobacterium, Agrobacterium tumefaciens infects plants and forms tumors referred to as crown galls. Agrobacterium rhizogenes infects plants to generate capillary roots. These are achieved by the transference of a region referred to as a T-DNA (transferred DNA) region on Ti plasmid or Ri plasmid to the plant at the time of infection and the incorporation into the plant genome. Accordingly, DNA which is to be incorporated into the plant genome is inserted into the T-DNA region on the Ti or Ri plasmid. When a plant host is infected by the bacteria belonging to the genus Agrobacterium, the DNA can be incorporated into the plant genome.

Tumor tissues, shoots, capillary roots, and the like resulting from the transformation can be used as such for cell culture, tissue culture, or organ culture. Also with the use of any conventionally known method of culturing plant tissues, plant bodies can be regenerated by the administration of suitably concentrated plant hormones, such as auxin, cytokinin, gibberellin, abscisic acid, ethylene, or brassinolide.

The gene of the present invention can be introduced into plants using a plant virus as a vector. Examples of plant viruses that can be used include the cauliflower mosaic virus. At the outset, a viral genome is inserted into an Escherichia coli-derived vector or others to prepare a recombinant. The gene of the present invention is then inserted into the viral genome. The thus-modified viral genome is cleaved out of the recombinant with a restriction enzyme and inoculated into a plant host. Thus, the gene of the present invention can be introduced into the plant host.

The recombinant vector of the present invention can be introduced not only into aforementioned plant hosts but also into, for example, bacteria belonging to the genus Escherichia such as Escherichia coli, the genus Bacillus such as Bacillus subtilis, or the genus Pseudomonas such as Pseudomonas putida, yeasts such as Saccharomyces cerevisiae or Schizosaccharomyces pombe, animal c ells such as COS cells or CHO cells, or insect cells such as Sf9. When bacteria such as Escherichia coli or yeast are used as hosts, the recombinant vector of the present invention is preferably capable of autonomous replication in the bacteria At the same time, the vector is preferably comprised of a promoter, a ribosome binding sequence, a transcription termination sequence, and the gene of the present invention. A gene that controls a promoter may also be contained therein.

Methods of introducing the recombinant vector of the present invention into bacteria are not particularly limited as long as DNA is introduced into the bacteria. Examples thereof include a method using calcium ions and the electroporation method, etc.

Methods of introducing the recombinant vector of the present invention into yeast are not particularly limited as long as DNA is introduced into the yeast. Examples thereof include the electroporation method, the spheroplast method, and the lithium acetate method, etc.

When animal cells are used as hosts, monkey COS-7 cell, Vero, Chinese hamster ovary (CHO) cell, mouse L cell, or the like is used. Methods of introducing the recombinant vector of the present invention into animal cells are not particularly limited as long as DNA is introduced into the animal cells. Examples thereof include the electroporation method, the calcium phosphate method, and the lipofection method, etc.

When insect cells are used as hosts, Sf9 cell or the like is used. Methods of introducing the recombinant vector of the present invention into insect cells are not particularly limited as long as DNA is introduced into the insect cells. Examples thereof include the calcium phosphate method, the lipofection method, and the electroporation method, etc.

Meanwhile, whether or not the gene of the present invention is incorporated into the host can be confimned by PCR, Southem hybridization, Northern hybridization, or the like. For example, DNA is prepared from the transformant, and a DNA-specific primer is designed and PCR is carried out. Subsequently, the amplification product is subjected to agarose gel electrophoresis, polyacrylamide gel electrophoresis, capillary electrophoresis, or the like and then stained with ethidium bromide, SYBR Green solution, or the like to detect the amplification product as a single band. Thus, the transformation can be confirmed. Alternatively, the amplification product can be detected by PCR using a primer previously labeled with a fluorescent dye, etc. Further, a method may be adopted in which the amplification product is bound to a solid phase such as a microtiter plate to confirm the amplification product by the fluorescent or enzyme reaction, etc.

The plant body of the present invention has a recombinant vector additionally having a companion cell-specific promoter located upstream of the gene of the present invention and has resistance against agents. The term “plant body” used herein refers to a whole plant that has been transformed with a recombinant vector additionally having a companion cell-specific promoter located upstream of the gene of the present invention. The plant body of the present invention can be obtained by introducing the recombinant vector into a plant cell or other, and regenerating the transgenic plant body from the resultant transformed plant cell. An example of a method for regeneration is one in which a callus-like transformed cell is transferred to a medium having a modified kind of hormone and a modified concentration thereof, and cultured therein to generate an adventitious embryo, thereby obtaining a complete plant body. Examples of media used include LS medium and MS medium, etc. The recombinant vector can be introduced into plant cells and the like in the same manner as described above.

In the plant body of the present invention, the sieve tube-localized GST protein that is encoded by the gene of the present invention is specifically expressed in the companion cell. Further, the expressed protein is transported to the sieve element. On the other hand, as described in the following examples, the sieve tube-localized GST protein of the present invention exhibits GST activity. GSH has been detected in the phloem sap of rice (Kuzuhara et al., Soil Sci. Plant Nutr. 46, 265-270, 2000). In the plant body of the present invention, accordingly, the sieve tubelocalized GST protein of the present invention, which is specifically expressed in the companion cell and then transported to the sieve element, allows GSH to conjugate with agents passing through the sieve tube, thereby detoxifying the same. Thus, the plant body according to the present invention can become resistant to agents. Examples of agents to which the plant body of the present invention can be made resistant include, but are not limited to, chloracetamide-based agents such as pretilachlor.

RPP31 is highly analogous to other type I GSTs. RPP31, however, contains regions that are not present in known type I GSTs (the regions are approximately between positions 126 and 177 in the amino acid sequence).

Functional significance of this sequence has not yet been elucidated. This sequence is, however, considered to function as a signal sequence for transporting the protein expressed in the companion cell to the sieve element.

Thus, the following is possible. The recombinant vector having the gene of the present invention and a foreign gene located downstream of the companion cell-specific promoter is introduced into the plant cell and the like. In this case, the gene of the present invention and the foreign gene are ligated to each other so as to encode a fusion protein. Subsequently, a transformed plant body is regenerated from the transformed cell.

In this plant body, the fusion protein that is encoded by the gene of the present invention and the foreign gene is specifically expressed in a companion cell with the aid of the companion cell-specific promoter. Subsequently, the fusion protein is transported to the sieve element with the aid of the signal sequence. Thus, the foreign protein can be localized as the fusion protein in the sieve tube. The foreign gene may encode any protein or peptide, and examples thereof include a gene encoding plant-derived insecticidal peptides or proteins. For example, the recombinant vector having, as a foreign gene, a gene encoding insecticidal peptides or proteins is introduced into a rice plant cell. Thus, sap-sucking insect-resistant genetic recombination rice, which does not express the insecticidal peptides or proteins in the seed but expresses them only in the sieve tube, can be produced.

### BRIEF DESCRIPTION OF THE DRAWINGS

FIG. 1 shows the two-dimensional electrophoresis pattern of a protein derived from phloem sap of rice.

FIG. 2 shows an alignment of the predicted amino acid sequence of RPP31 and amino acid sequences of other type I plant GSTs.

FIG. 3A shows CBB staining of SDS-PAGE gel in which the recombinant RPP31 produced in Escherichia coli was electrophoresed.

FIG. 3B shows GST activity of recombinant RPP31 produced in Escherichia coli.

FIG. 3C shows GST activity of phloem sap of rice.

FIG. 4A shows the result of Western blot analysis on phloem sap and leaf extract of rice.

FIG. 4B shows the result of Western blot analysis on leaf extract and root extract of rice.

FIG. 5A is a full photograph of the cross-sectioned slice of the base of the leaf sheath, which has been immunohistologically stained.

FIG. 5B is an enlarged photograph corresponding to the position “B” in FIG. 5A.

FIG. 5C is an enlarged photograph corresponding to the position “C” in FIG. 5A.

FIG. 5D is an enlarged photograph corresponding to the position “D” in FIG. 5A.

FIG. 5E is an enlarged photograph corresponding to the position “E” in FIG. 5A.

FIG. 5F is an enlarged photograph corresponding to the position “F” in FIG. 5A.

FIG. 5G is a photograph showing a region of a large fibrovascular bundle stained using an immunologically absorbed antibody as a negative control.

FIG. 6 shows the result of Western blot analysis on RPP31 in a leaf extract and a root extract derived from rice treated with pretilachlor and/or fenclorim.

### BEST MODE FOR CARRYING OUT THE INVENTION

### EXAMPLES

The present invention is hereafter described in more detail with reference to the examples. The technical scope of the present invention, however, is not limited to these examples.

Supplier of Reagents for Research

The reagents used in the following examples were products of Wako Pure Chemical Industries, Ltd. (Osaka, Japan) unless otherwise specified.

### Example 1

### Identification of Sieve Tube-Localized GST

(1) Recovery of Phloem Sap from rice Plant

As described in Nakamura et al., Plant Cell Physiol. 34, 927-933, 1993, a rice plant (Oryza sativa L. cv. Kantou) was allowed to grow in a temperature-controlled greenhouse (30/25° C., day/night) under hydroponic conditions. Subsequently, phloem sap of rice was recovered from the leaf sheath of 4 to 5 week-old rice plants by the method using cut ends of stylets of rice Brown Planthoppers (Kawabe et al., Plant Cell Physiol. 21, 1319-1327, 1980). All procedures of recovering phloem sap were carried out indoors under indoor light conditions (20 μmol s⁻¹ m⁻²) at 25° C. at a relative humidity of 60%. The recovered phloem sap was stored at −20° C. until the analysis. The phloem sap to be used in assaying GST activity was stored at 4° C.

(2) Analysis of N-termiinal Amino Acid Sequence of RPP31

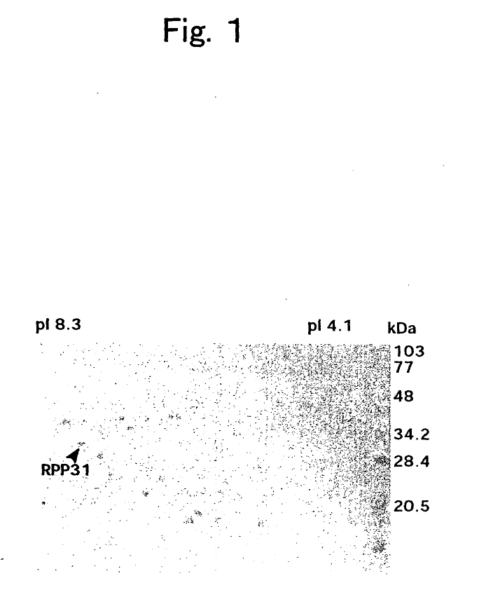

Phloem sap of rice (300 μl) was centrifuged at 15,000 rpm for 20 minutes. Subsequently, the precipitated protein was subjected to two-dimensional polyacrylamide gel electrophoresis (2D-PAGE) as described in Nakamura et al., Plant Cell Physiol. 34, 927-933, 1993. After being separated on gel, the protein was electroblotted onto polyvinylidene difluoride (PVDF) membrane (Immobilon, Millipore Co., USA). Subsequently, the PVDF membrane was stained using a Coomassie brilliant blue G-250 (CBB) buffer (0.025% (w/v) CBB, 10% (v/v) acetic acid, and 40% (v/v) methanol). The results are shown in FIG. 1. In FIG. 1, the portion pointed to by an arrow represents a 31 kDa protein.

A spot of the 31 kDa protein was cut off from the PVDF membrane. Thereafter, the N-terminal amino acid sequence of the protein was analyzed using the gas phase protein sequencer (492 HT, Applied Biosystems, USA). As a result, the N-terminal amino acid sequence of the protein was found to be PGAVKVFGSP (SEQ ID NO: 4).

Further, a nucleotide sequence deduced from the N-terminal amino acid sequence was searched for using the DNA database (DDBJ, http://www.ddbj.nig.acjp). Based on this search, two kinds of EST clones of rice (O. sativa, cv. Nipponbare), i.e., S13415 (Accession No. D47745) and R2087 (Accession No. D24517), and a BAC clone of rice (O. sativa, cv. Nipponbare), OSJNBb0015I11 (Accession No. AC051633-22), were identified as clones comprising the gene which encodes the protein having the N-terminal amino acid sequence.

(3) Analysis of DNA Sequence of EST Clone

The whole nucleotide sequences of each EST clone were determined using the EST clones, i.e., S13415 (Accession No. D47745) and R2087 (Accession No. D24517), four kinds of primers corresponding to both EST clones, and a sequence kit, BigDye Terminator (PerkinElmer Biosystems, USA) with a sequence analyzer (ABIPRISM™310, PerkinElmer Biosystems, USA). Both EST clones were provided from the National Institute of Agrobiological Sciences (http://rgp.dna.affrc.go jp).

Sequences of four kinds of primers corresponding to both EST clones were as shown below.

5′- GCAATFAACCCTCACTAAAGGG -3′ (SEQ ID NO: 5)

5′- CGTAATACGACTCACTATAGGGC -3′ (SEQ ID NO: 6)

5′- CGTTGGAGAGGTCGTCGATCGA -3′ (SEQ ID NO: 7)

5′- GGAGACGTCGGCCCACCACCGG -3′ (SEQ ID NO: 8)

As a result of the aforementioned sequence analysis, nucleotide sequences of the EST clones, i.e., S13415 (Accession No. D47745) and R2087 (Accession No. D24517), were determined as shown in SEQ ID NO: 2 and SEQ ID NO: 3, respectively. Based on this analysis, it was ascertained that both clones contained the gene which encoded the same protein having an estimated molecular weight of 30.7 kDa, which was equivalent to the molecular weight of RPP31. Based on the DNA sequence of the BAC clone, OSJNBb0015I11 (Accession No. AC051633-22), the gene encoding RPP31 was found to contain 3 exons and 2 introns. This genetic structure is consistent with type I plant GSTs. Further, with the use of the database on rice (O. sativa L. ssp. Indica) contig of the whole genome shotgun (Beijing Genomics Institute, http://btn.genormics.org.cn/rice), nucleotide sequences deduced from the N-terminal amino acid sequence were searched for. Based on the search, the rice genome was found to contain one copy of the gene encoding-a protein containing the N-terminal amino acid sequence.

(4) Search and Analysis of Homology

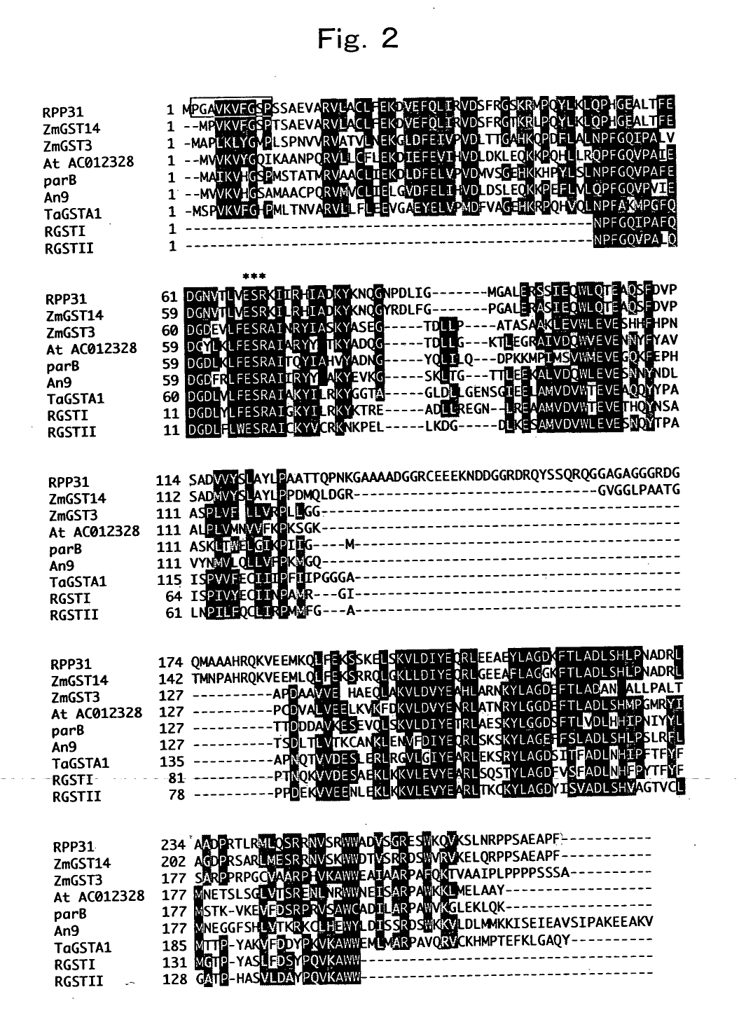

Homology between RPP31 and type I plant GSTs at the amino acid level was searched for and analyzed (DDBJ;http://www.ddbj.nig.ac.jp) (FIG. 2). Multiple sequence alignment was performed using the GenomeNet Clustal W (http://clustalw.genome.ad.jp/). The results are shown in FIG. 2.

The following type I plant GSTs were compared: maize (Z. mays)-derived ZmGST14 and ZmGST3 (ZmGST14: Accession No. AF244679, McGonigle et al., Plant Physiol. 124, 1105-1120, 2000, ZmGST3: Accession No. AJ010295-1, Grove et al., Nucleic Acid Res. 16, 425-428,1988); A. thaliana-derived At AC012328 (Accession No. AC012328-12); tobacco plant (N. tabacum)-derived parB (Accession No. D10524-1, Takahashi and Nagata, Proc. Natl. Acad. Sci. USA 89, 56-59, 1992); petunia (Petunia X hybrida)-derived An9 (Accession No. Y07721-1, Alfenito et al., Plant Cell 10, 1135-1149, 1998); wheat (T. aestivum)-derived TaGSTA1 (Accession No. T06509, Dudler et al., Mol. Plant-Microbe Interact. 4, 14-18, 1991); and rice (O. sativa)-derived RGSTI and RGSII (partial sequences of GST) (Accession No. AJ002380, AJ002381, Wu et al., Physiol. Plant. 105, 102-108, 1999).

In FIG. 2, the amino acid sequence within the white flame represents the N-terminal amino acid residues that were detected by the protein sequencer. The same residues and analogous residues were shadowed black and gray, respectively, using the ISREC BOXSHADE Software (http://www.ch.embnet.org/software/BOX#form.html). Asterisks (*) indicate amino acids conserved in the GSH binding site (G-site) (McGonigle et al., Plant Physiol. 124, 1105-1120, 2000). Dashes (-) indicate gaps.

As shown in FIG. 2, RPP31 was highly analogous to type I plant GSTs. For example, RPP31 has a Glu-Ser-Arg triplet (amino acid residues marked with asterisks (*) in FIG. 2). This triplet is highly conserved in the GSH binding site (G-site) of type I plant GSTs. RPP31 was most analogous to ZmGST14 (70.5% amino acid sequence identity according to FASTA search). Accordingly, it is considered that RPP31 may be classified as type I plant GST. Unlike other type I GSTs, however, RPP31 contained regions that were not present in known type I GSTs (the regions are approximately between positions 126 to 177 in the amino acid sequence). Further comparison of N-terminal amino acid sequences between RPP31 and other type I plant GSTs revealed that the RPP31 protein did not contain methionine at the N-termiinus.

### Example 2

### In Vitro GST Enzyme Assay

(1) Synthesis of Recombinant RPP31

Recombinant RPP31 to be used in the GST enzyme assay was synthesized in Escherichia coli. At the outset, cDNA of RPP31 was amplified by PCR using the rice EST clone S13415 (Accession No. D47745, SEQ ID NO: 2) as a template and primers corresponding to the clone.

Conditions for PCR and primer sequences were as shown below.

Conditions for PCR: 30 cycles of at 96° C. for 45 seconds, at 60° C. for 45 seconds, and at 72° C. for 2 minutes, and then storage at 4° C.

Primer 617:5′-AGAATTCCATATGCCGGGAGCCGTGAAGGT -3′ (SEQ ID NO: 9)

Primer 428: 5′-ATGCCATGGTCAGAAGGGAGCCTCGGCGG -3′ (SEQ ID NO: 10)

Subsequently, the amplified cDNA fragment of RPP31 was cleaved with restriction enzymes NdeI and NcoI, and inserted into a plasmid vector pET30b (Novagene Co., Darmstadt, Germany) which had been cleaved with the same restriction enzymes for subcloning. The obtained plasmid is hereafter referred to as “pAF27.”

Subsequently, pAF27 was introduced into Escherichia coli strain BL21 DE3 (Novagene Co., Darmstadt, Germany), and the RPP31 protein was synthesized in the following manner. First, pAF27-containing Escherichia coli was precultured in LB medium at 26.5° C. overnight. This preculture solution (1 ml) was then added to 50 ml of LB medium, and culture was further conducted at 26.5° C. for 2 hours. Thereafter, isopropylthio-β-D-galactoside (IPTG) was added to a final concentration of 0.1 M to induce protein synthesis, and culture was further conducted at 26.5° C. for 4 hours. The culture solution was then centrifuged at 12,000 rpm for 10 minutes to recover Escherichia coli. An extraction buffer (0.5 ml, 20 mM Tris-HCI (pH 7.8) and 0.2 M NaCi) was added to the resulting Escherichia coli and dissolved therein by ultrasonication. Centrifugation was then carried out at 12,000 rpm for 10 minutes to remove cell debris. The resultant supematant was used as an extract in the assay of GST activity.

(2) Confirmation of Recombinant RPP31 Expression Induced by IPTG

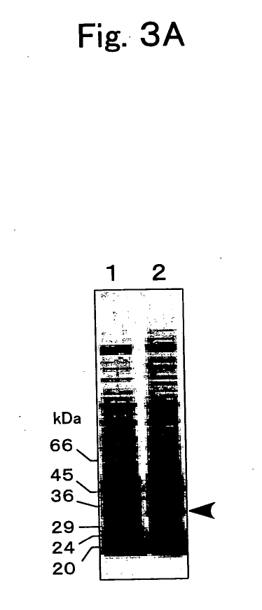

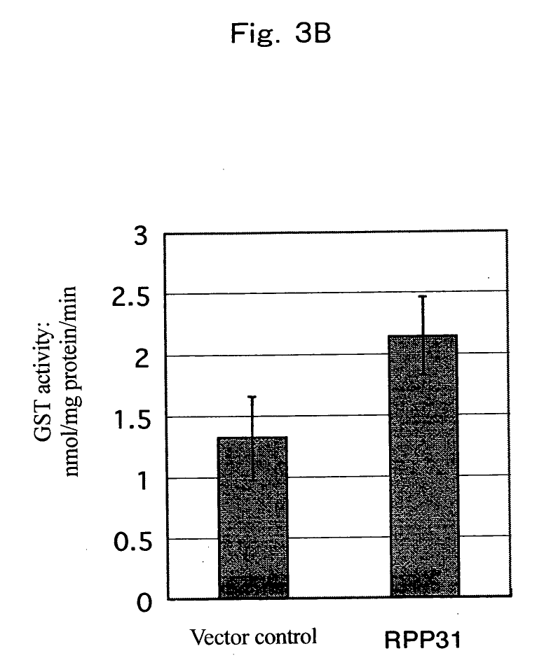

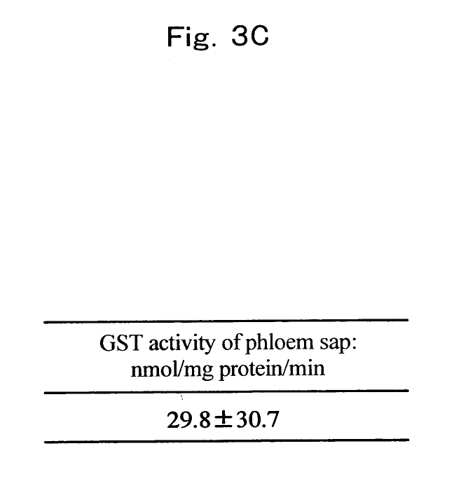

SDS electrophoresis and CBB staining were carried out in order to confirm whether or not recombinant RPP31 was expressed in the IPTG-treated Escherichia coli. The protein extracted before IPTG induction and the protein extracted after IPTG induction, derived from pAF27-containing Escherichia coli, were subjected to SDS electrophoresis. A gel was then stained using a CBB buffer (0.025% (w/v) CBB, 10% (v/v) acetic acid, and 40% (v/v) methanol). The results are shown in FIG. 3A. In FIG. 3A, Lane 1 indicates the protein extracted before IPTG induction and Lane 2 indicates the protein extracted after IPTG induction. An arrow indicates the RPP31 protein.

As shown in FIG. 3A, it could be confirmed that the RPP31 was induced to express after IPTG induction.

(3) Assay of GST Activity on Recombinant RPP31

GST activity of the extract derived from pAF27-containing Escherichia coli was assayed using a general GST substrate, 1-chloro-2,4-dinitrobenzene (CDNB). In the assay, the extract derived from pAF27-containing Escherichia coli and an extract derived from pET30b-containing Escherichia coli (vector control) were used. Enzyme reaction was conducted for 5 minutes using 0.5 ml of reaction solution comprising 150 μl of mixture of an extract and water, and a reaction buffer (50 L of 10 mM CDNB, 50 μl of 10 mM GSH, and 250 μl of 0.2M KH₂PO₄ (pH 6.5)). The CDNB-GSH conjugate has an absorption peak at 340 nm. Thus, an increase in the absorbance at 340 nm was assessed as GST activity. Details of the method for assaying GST activity were in accordance with “Kassei Sanso Jikken Purotokoru (Protocol for Active Oxygen Experiment)” (Naoyuki Taniguchi (ed.), Shujunsha). The results are shown in FIG. 3b.

Based on the molar absorption coefficient of the CDNB-GSH conjugate, i.e., 9,600, the enzyme activity level of the protein in the reaction solution was assayed. The graph in FIG. 3b shows the average and the standard deviation obtained from the experiment which was repeated four separate times. In comparison with the extract derived from pET30b-containing Escherichia coli (vector control), the extract derived from pAF27-containing Escherichia coli exhibited a significant increase in CDNB-GSH conjugation activity, i.e., GST activity (t test, p<0.005). This indicates that recombinant RPP31 has GST activity.

(4) Assay of GST Activity of the Phloem Sap of Rice

GST activity of the phloem sap of rice was assayed in order to investigate whether or not the phloem sap of rice per se had CDNB-GSH conjugation activity. With the use of an ultrafiltration filter Microcon YM-3 (Millipore Co., Tokyo, Japan), 60 μl of phloem sap of rice was concentrated to about 3 μl at 4° C. In the same manner as above, CDNB was used as a. substrate to assay GST activity of concentrated phloem sap based on a change in the absorbance at 340 nm. In order to assay the absorbance using 5 μl of reaction solution (0.5 μl of 10 mM CDNB, 0.5 μl of 10 mM GSH, 2.5 μl of 0.2M KH₂PO₄ (pH 6.5), 0.5 μl of water, and 1 μl of concentrated phloem sap), a microcell was used (inner diameter: 0.5 mm, Absorbance Capillary Adaptor Cell; Helix Co., CA, USA). A low molecular weight fraction (molecular weight: <3,000), which was passed through the filter when concentrating the phloem sap, and synthetic phloem sap, which has a similar composition to the phloem sap (5 mM Ca (NO₃)₂, 10 mM K₂SO₄, 10 mM K₂HPO₄, 20 mM KCI, 10 mM MgCl₂,5 mM HCl, 10 mM KOH, 10 mM maleate, 20 mM aspartic acid, 20 mM glutamic acid, 20 mM serine, 20 mM threonine, 10 mM tyrosine, 10 mM lysine, 10 mM phenylalanine, 5 mM tryptophan, and 250 mM sucrose), were used as negative controls. Measurement was independently carried out twice. The results are shown in FIG. 3C.

The absorbance changes of negative controls, i.e., a low molecular weigh fraction of phloem sap and synthetic phloem sap, did not differ from the background measured using water. In comparison with these, more significant absorbance change was detected in the concentrated phloem sap containing RPP31. This indicates that RPP31 has GST activity in the sieve tube of rice plants.

### Example 3

### Western Blot Analysis

(1) Synthesis of His-tagged RPP31

His-tagged RPP31 was prepared as an antigen in the following manner in order to prepare an antibody to RPP31. cDNA ofRPP31 was first amplified by PCR using rice EST clone S13415 (Accession No. D47745, SEQ ID NO: 2) as a template and primers corresponding to the clone.

Conditions for PCR and primer sequences were as shown below.

Conditions for PCR: 30 cycles of at 96° C. for 45 seconds, at 60° C. for 45 seconds, and at 72° C. for 2 minutes, and then storage at 4° C.

Primer 427: 5′-CGGGGTACCGACGACGACGACAAGATGCCGGGAGCCGTGAA -3′ (SEQ IDNO: 11)

Primer 428: 5′-ATGCCATGGTCAGAAGGGAGCCTCGGCGG -3′ (SEQ ID NO: 10)

The amplified fragment was then treated using restriction enzymes KpnI and NcoI. Thereafter, a restriction enzyme-treated fragment was inserted downstream of the His-tag sequence of a plasmid vector pET30b which had been cleaved with the same restriction enzymes for subcloning. In the same manner as described concerning pAF27 in Example 1, a supernatant of Escherichia coli extract containing His-tagged recombinant RPP31 was then prepared. In accordance with the manufacturer's instruction, His-tagged RPP31 protein was purified in a nickel column using a His-binding buffer kit (Novagene Co., Darmstadt, Germany).

(2) Production and Purification of Rabbit Anti-RPP31 Polyclonal Antibody

A rabbit was immunized with 3.0 mg of His-tagged RPP31 protein as an antigen. Production of anti-RPP31 serum was consigned to Sawady Technology (Tokyo, Japan). Subsequently, IgG in the immunized rabbit serum was purified using the ImmunoPure IgG (protein A) purification kit (PIERCE Co., Rockford, USA). In accordance with the method of Yamaya et al., Plant Physiol. 100, 1427-1432, 1992, the anti-RPP31 IgG was then subjected to affinity purification in the following manner.

The His-tagged RPP31 protein (100 μg) was subjected to SDS electrophoresis and transferred onto a nylon membrane. Subsequently, the nylon membrane was stained with a Ponceau S Staining Solution (1% (v/v) acetic acid containing 0.1% (w/v) Ponceau S (SIGMA CHEMICAL, MO, USA)) and washed with 1% (v/v) acetic acid and deionized water, and a band of RPP31 was cleaved out. The band was washed with 100 μM NaOH to completely remove Ponceau S. The band was washed three times with deionized water and with PBS (137 mM NaCl, 2.7 mM KCl, 8.1 mM Na₂HPO₄, and 1.5 mM KH₂PO₄), and a RPP31 binding sheet was prepared. The binding sheet was blocked for 4 hours in TPBS (PBS containing 0.1% (w/v) Tween 20) containing 5% (w/v) skimmed milk (Santa Cruz Biotechnology, USA) (TPBS-MILK). Subsequently, the binding sheet was transferred into 9 ml of PBS-BSA (PBS containing 0.3% (w/v) BSA and 0.05% (w/v) NaN₃) containing 450 μl of IgG solution and incubated overnight. Thus, IgG was allowed to bind to the His-tagged RPP31. Thereafter, the binding sheet was washed three times with TPBS-MILK for 20 minutes and once with PBS for 10 minutes. Further, the binding sheet was washed for 2 minutes and 30 seconds in 500 μl of 0.2 M glycine-HCl (pH 2.5) to elute IgG. Immediately, 85 μl of Tris-HCl (pH 8.8) containing 6.9% (w/v) BSA was added to neutralize the eluate. The binding sheet was used again to purify this anti-RPP31 IgG-containing eluate. The thus obtained eluate was used in the succeeding Western blot analysis and immunohistological staining experiment as an affinity purified RPP31 antibody. The solution which had been incubated with the binding sheet was incubated with the binding sheet two more times in order to completely remove anti-RPP31 IgG. As an immunologically absorbed antibody, this solution was used in the immunohistological staining experiment as a negative control.

(3) Extraction of Soluble Proteins from Leaf and Root of Rice

A leaf or root of rice was ground in liquid nitrogen, and, in the case of a leaf, mixed with an equivalent amount of, and in the case of a root, a half amount of extraction buffer (25 mM Tris-HCl (pH 6.8), 5% β-mercaptoethanol, 5 mM P-APMSF, and 25 μg/ml leupeptin). The thus obtained homogenates were centrifuged twice at 13,000 g at 4° C. for 30 minutes to obtain an extract containing soluble proteins, respectively. Total protein concentrations of the leaf extract, the root extract, the phloem sap, and the Escherichia coli cell extract were measured using the Protein Assay Reagent (BIO-RAD Co., CA, USA). The measuring method was in accordance with the attached protocol.

(4) Western Blot Analysis

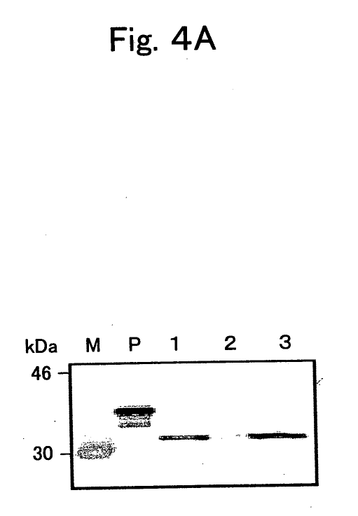

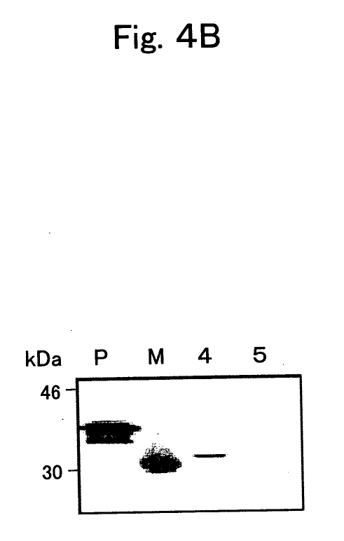

RPP31s in the leaf extract, the root extract, and the phloem sap of rice were detected by Western blot analysis. Escherichia coli cell-derived His-tagged RPP31 was used as a positive control. At the outset, the leaf extract, the root extract, and the phloem sap were subjected to SDS electrophoresis and then transferred onto a PVDF membrane. Subsequently, the PVDF membrane was blocked in 3% (w/v) gelatin (BIO-RAD Co., CA, USA). Immunoblotting was then carried out using the affinity purified anti-RPP31 antibody as a primary antibody and a goat anti-IgG (H+L) horseradish peroxidase conjugate (BIO-RAD Co., CA, USA) as a secondary antibody. Diaminobenzidine was used as a substrate for detection. The results are shown in FIGS. 4A and 4B.

In FIG. 4A, M indicates a molecular weight marker, P indicates His-tagged RPP31 (positive control, 50 ng), Lane 1 indicates phloem sap (protein content: 1.5 μg), Lane 2 indicates a leaf extract (protein content: 1.5 μg), and Lane 3 indicates a leaf extract (protein content: 15 μg).

As shown in FIG. 4A, a 31 kDa major band corresponding to the molecular weight of RPP31 was detected in the phloem sap and the leaf extract of rice plants. Three thin bands were further detected at approximately 37 kDa, 35 kDa, and 28 kDa positions of the phloem sap. Since many of plant GSTs have molecular masses of 25 to 29 kDa (Marrs et al., Annu. Rev. Plant Physiol. Plant Mol. Biol.47,127-158,1996), the 28 kDa band could be a different GST. Further, two bands, the approximately 37 kDa and the 35 kDa bands were significantly different from the molecular weight of GST and thus, it was considered that they were detected nonspecifically by the antibody.

On the other hand, the band of RPP31 in the phloem sap was denser than that of the leaf extract prepared by electrophoresing an equivalent amount of protein. Accordingly, it can be considered that the abundance of RPP31 in the phloem sap is high.

On the other hand, in FIG. 4B, P indicates His-tagged RPP31 (positive control, 50 ng), M indicates a molecular weight marker, Lane 4 indicates a leaf extract (protein content: 10 μg), and Lane 5 indicates a root extract (protein content: 10 μg).

As shown in FIG. 4B, RPP31 was not detected in the root extract having the same protein content as the leaf extract. This indicates that the quantity of RPP31 present in the root is lower than the detection limit.

### Example 4

### Confirmation of RPP31 Localization by Immunohistological Staining Method

Cellular localization of RPP31 was investigated in the cross sectioned slice of the base of leaf sheath of rice plants. At the outset, approximately 5 mm of the base of leaf sheaths were cut off from the 5-week old rice plants and immobilized in a fixing buffer (1.85% (v/v) formaldehyde, 45% (v/v) ethanol, and 5% (v/v) acetic acid). Subsequently, the tissue was dehydrated with t-butyl alcohol and embedded in paraffin. Details of the method of paraffin embedding were in accordance with the method of Kouchi and Hata, Mol. Gen. Genet. 238: 106-119, 1993. The tissue was then prepared into 10 μm-thick cross-sectioned slices using a microtome (LR-85, YAMATO KOHKI Industrial Co., Japan) and then transferred onto glass slides. Thereafter, in accordance with the manufacturer's instruction, RPP31 on the surface of the tissue section was visualized using the Vectastain ABC Elite Kit (Vector Lab. Inc., CA, USA) and the aforementioned affinity purified rabbit anti-RPP31 polyclonal antibody. The aforementioned immunologically absorbed antibody was used as a primary antibody of the negative control.

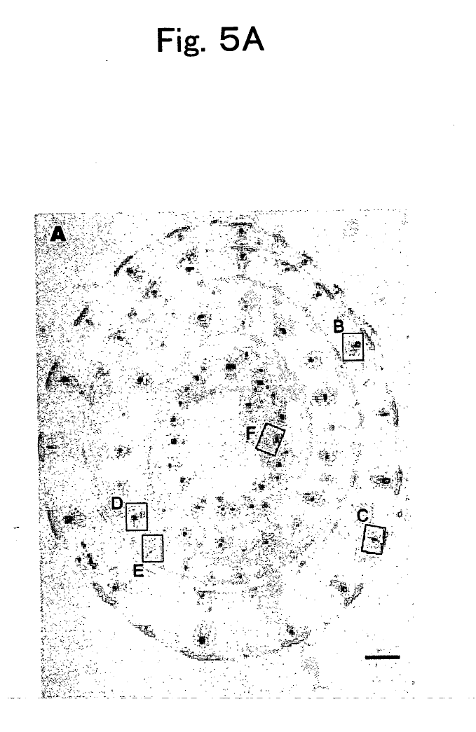

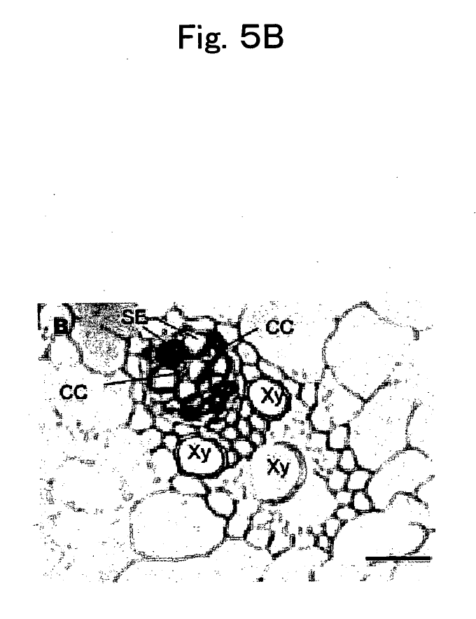

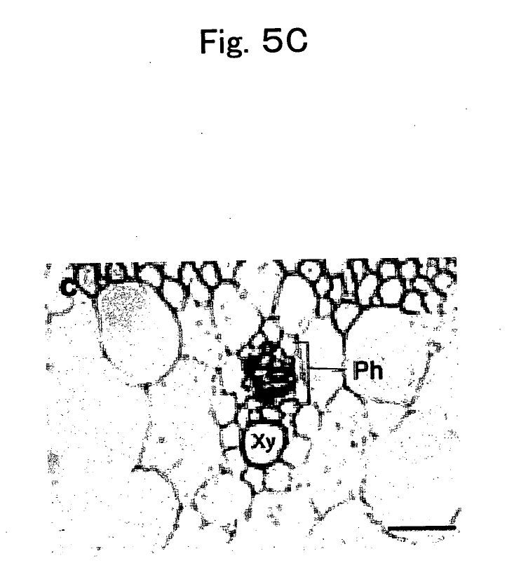

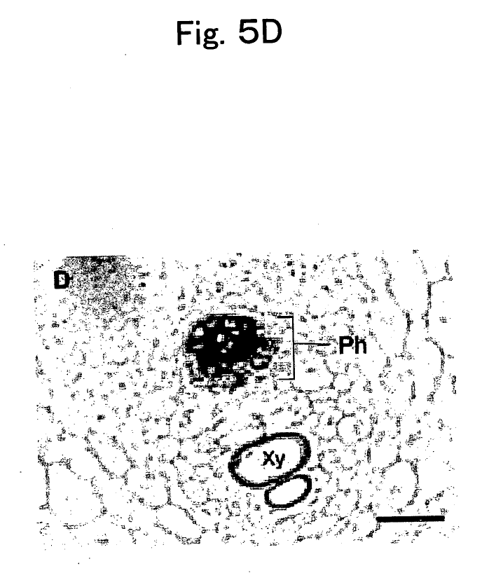

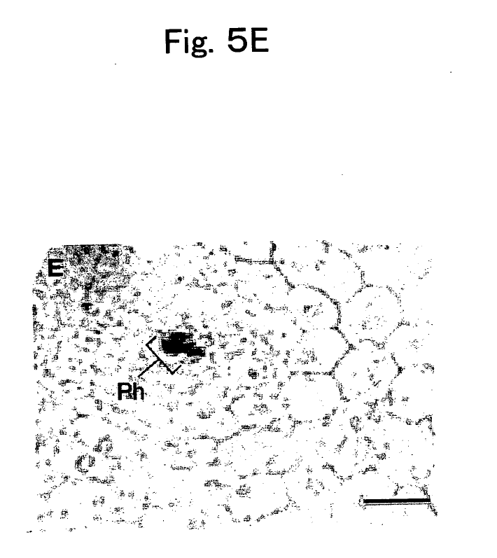

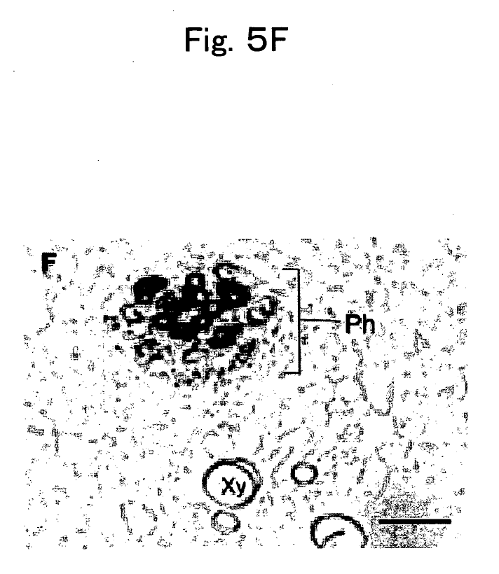

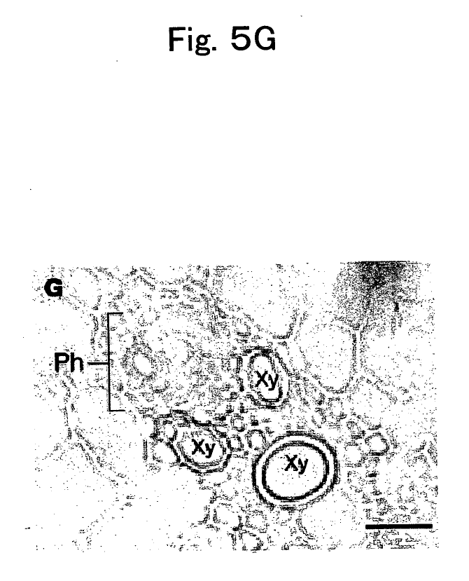

FIG. 5A is a full photograph of the cross-sectioned slice of the base of the leaf sheath, which has been immunohistologically stained. A scale bar is equal to 200 μm. Since one field of view of a microscope cannot cover an entire slice, 6 separate fields of photographs were taken and then pieced together using the Photoshop software (Adobe System, CA, USA). An immature leaf located at the innermost position is referred to as “P1” (Nemoto et al., Crop Sci 35:24-29, 1995). Leaves are outwardly numbered P2 and P3. As shown in FIG. 5A, RPP31 signals were detected only in the phloem of the fibrovascular bundle but were not detected in the mesophyll cells or epidermal cells.

FIGS. 5B to 5F are enlarged photographs corresponding to each of the positions shown in FIG. 5A. Abbreviations used in the drawings are defined as follows: Xy: xylem vessel; CC: companion cell; SE: sieve element; and Ph: phloem. In FIGS. 5B to 5F, the scale bar is equal to 25 μm.

FIGS. 5B and 5C are enlarged photographs of a large fibrovascular bundle and a small fibrovascular bundle of the leaf P3, respectively. In the large fibrovascular bundle and the small fibrovascular bundle of the leaf P3, RPP31 was localized in the sieve element and the companion cell, however, it was not localized in the xylem or in the vascular bundle sheath cell. In the large fibrovascular bundle of the leaf P3 in which the companion cell is particularly clearly distinguished from the sieve element, signals were strong in the sieve element but weak in the companion cell (FIG. 5B). This indicates that RPP31 is transported from the companion cell to the sieve element.

FIGS. 5D and 5E are respectively enlarged photographs of a large fibrovascular bundle and a small fibrovascular bundle of the leaf P2. Similarly, in the leaf P2, RPP31 was localized in the phloem but was not localized in the xylem. Further, FIG. 5F is an enlarged photograph of the fibrovascular bundle of the leaf P1. Similarly, also in the leaf P1, RPP31 was localized in the phloem.

FIG. 5G is a photograph showing a region of a large fibrovascular bundle stained using an immunologically absorbed antibody as a negative control.

These results demonstrate that RPP31 is localized in the companion cell and in the sieve element of the leaf of rice plants.

In this experiment, there were a sieve element in which signals were observed in the whole cell and a sieve element in which signals were observed near the cell membrane but were not observed in the center of the cell. This could be because cell contents were not immobilized but discharged in the sieve element in which signals were not observed in the center of the cell. In the sieve element in which signals were observed in the whole cell, cell contents remained because, for example, cells were cleaved out around the sieve plate.

### Example 5

### Investigation on RPP31's Inducibility to Agents

Two types of type I GST genes isolated from rice, i.e., RGSTI and RGSTII, are induced to express by herbicides pretilachlor and safener fenclorim (Wu et al., Physiol. Plant. 105, 102-108, 1999). In order to investigate whether or not RPP31 is induced by treatment with pretilachlor and fenclorim, extracts of leaves and roots of plants containing soluble proteins treated with the agents were analyzed by Western blotting.

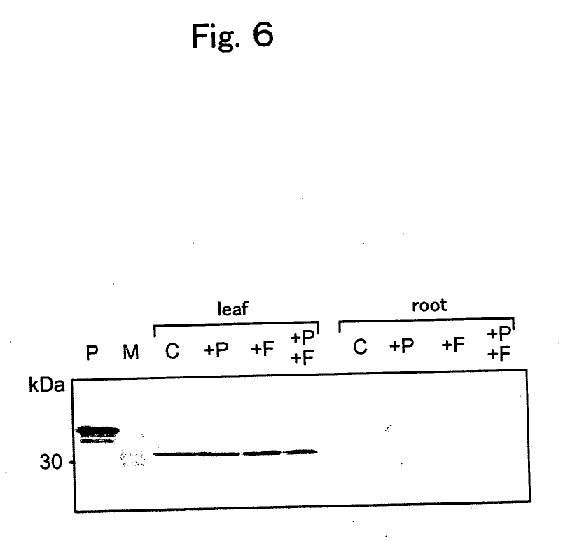

As described in Example 1, rice (O. sativa cv. Kantou) was first allowed to grow for 3 to 4 weeks. Subsequently, four rice plants at the fifth- to seventh-leaf stages were transferred into 500 ml of hydroponic medium containing 2×10⁻⁵ M pretilachlor (Kantou Chemical Co., Japan), 2×10⁻⁵ M fenclorim (Ciba-Geigy Co., Japan), or both thereof, and root portions of rice were immersed in agent-containing hydroponic medium for 1 day. As described in Example 3, soluble proteins in the leaf and in the root were then extracted, and the obtained leaf and root extracts were used to conduct Western blot analysis. The results are shown in FIG. 6.

In FIG. 6, P indicates His-tagged RPP31 (positive control, 50 ng), M indicates a molecular weight marker, C indicates a leaf or root extract (protein content: 10 μg) derived from untreated plants, +P indicates a leaf or root extract (protein content: 10 μg) derived from the pretilachlor-treated plant, +F indicates a leaf or root extract (protein content: 10 μg) derived from the fenclorim-treated plant, and +P+F indicates a leaf or root extract (protein content: 10 μg) derived from the plants treated with both of pretilachlor and fenclorim.

After the treatment with pretilachlor, fenclorim, or both thereof, the RPP31 content in the leaf extract did not change. Even after the treatment with pretilachlor or/and fenclorim, RPP31 was not detected in the root extract. In the rice plant which had been treated with pretilachlor or/and fenclorim for 10 days, the RPP31 content in the leaf extract did not change as in the case of the treatment for 1 day, and no RPP31 was detected in the root extract (data not shown). These results indicate that RPP31 is not induced by the treatment with pretilachlor or/and fenclorim in rice.

According to the above examples, it was found that RPP31 was not localized in the root but was localized in the sieve element and in the companion cell of the leaf. Further, RPP31 is not induced to express by treatment with pretilachlor or/and fenclorim. Thus, RPP31 was found to be the GST that constantly exists in the leaf.

Industrial Applicability

The present invention provides a sieve tube-localized glutathione S-transferase. Accordingly, the present invention is considered to facilitate the elucidation of the functional mechanism of GST in the sieve tube.

1. A gene encoding a protein described in the following (a) or (b):
(a) a protein consisting of the amino acid sequence as shown in SEQ ID NO: 1; or
(b) a protein consisting of the amino acid sequence having the substitution, deletion, or addition of at least one amino acid in the amino acid sequence as shown in SEQ ID NO: 1 and having glutathione S-transferase activity.

2. A sieve tube-localized glutathione S-transferase protein that is encoded by the gene according to claim 1.

3. A recombinant vector having the gene according to claim 1.

4. The recombinant vector according to claim 3 additionally having a companion cell-specific promoter located upstream of the gene.

5. A transformant having the recombinant vector according to claim 3.

6. A plant body that has the recombinant vector according to claim 4 and that has resistance against agents.
